(19) United States
(12) Patent Application Publication
(10) Pub. No.: US 2023/0036182 A1
(43) Pub. Date: Feb. 2, 2023

(54) INFORMATION PROCESSING APPARATUS, INFORMATION PROCESSING METHOD, AND INFORMATION PROCESSING PROGRAM

(71) Applicant: SONY GROUP CORPORATION, Tokyo (JP)

(72) Inventors: Takashi OGATA, Tokyo (JP); Yasunori KAMADA, Kanagawa (JP); Kenji SUZUKI, Tokyo (JP)

(73) Assignee: SONY GROUP CORPORATION, Tokyo (JP)

(21) Appl. No.: 17/791,691

(22) PCT Filed: Jan. 8, 2021
(86) PCT No.: PCT/JP2021/000511

§ 371 Date: Jul. 8, 2022

(30) Foreign Application Priority Data
Jan. 17, 2020 (JP) 2020-005939

(51) Int. Cl. G06V 40/12; G06V 40/60; G06V 40/50

(52) U.S. Cl. CPC G06V 40/1365; G06V 40/67; G06V 40/1335; G06V 40/50

(57) ABSTRACT

There is provided an information processing apparatus including a processing unit that performs living body-related processing on the basis of an image of a part of a living body of a user, the image being generated in a state in which the part of the living body is in contact with a contact surface, and contact information acquired in the state in which the past of the living body is in contact with. the contact surface.

### TECHNICAL FIELD

The present technology relates to an information processing apparatus, an information processing method, and an information processing program.

### BACKGROUND ART

In recent years, biometric authentication using a part of a user's living body has been used as a method of authenticating the user on a smartphone, a wearable device, or the like. The biometric authentication includes, for example, fingerprint authentication using a fingerprint of a. finger. The fingerprint is a pattern. having raised. lines (ridges) in the skin of a fingertip where the openings of sweat glands lie, and the shape of the fingerprint varies from person to person and is invariable throughout life. Therefore, an authentication processing apparatus using a fingerprint that can be used as information for identifying and authenticating individual users has been proposed (Patent Document 1).

### CITATION LIST

### Patent Document

Patent Document 1: Japanese Laid-Open Patent Publication No. 2017-196319

### SUMMARY OF THE INVENTION

### Problems to be Solved by the Invention

In the fingerprint authentication, there are registration processing of registering a fingerprint as authentication information and authentication processing of checking whether the registered fingerprint matches a fingerprint of a user who uses a device 100 or the like. The registration processing and the authentication processing are performed when the user presses his/her finger against a fingerprint sensor.

However, the user does not necessarily press the fingerprint sensor at the same degree of pressing between at the time of registration and at the time of authentication, and furthermore, positions of the finger to be brought into contact are not necessarily the same. There is a problem that the accuracy of the fingerprint authentication is affected when a contact mode such as the degree of pressing or the position of the finger to be brought into contact is different between at a registration stage and at an authentication stage.

The present technology has been made in view of such a point, and an object thereof is to provide an information processing apparatus, and information processing method, and an information processing program capable of performing highly accurate biometric authentication even when a contact mode of a part of a living body is different between at the time of registration and at the time of authentication.

### SOLUTIONS TO PROBLEMS

In order to solve the problem described above, a first technology is an information processing apparatus including a processing unit that performs living body-related processing on the basis of an image of a part of a living body of a user, the image being generated in a state in which the part of the living body is in contact with a contact surface, and contact information acquired in the state in which the part of the living body is in contact with the contact surface.

Furthermore, a second technology is an information processing method of performing living body-related processing on the basis of an image of a part of a living body of a user, the image being generated in a state in which the part of the living body is in contact with a contact surface, and contact information acquired in the state in which the part of the living body is in contact with the contact surface.

Moreover, a third technology is an information processing program that causes a computer to execute an information processing method for performing living body-related processing on the basis of an image of a part of a living body of a user, the image being generated in a state in which the part of the living body is in contact with a contact surface, and contact information acquired in the state in which the part of the living body is in contact with the contact surface.

### BRIEF DESCRIPTION OF DRAWINGS

FIG. 1 is a block diagram illustrating a configuration of a device 100.

FIG. 2 is a view illustrating arrangement of an imaging sensor 107 and a pressing sensor 108 in a first embodiment.

FIG. 3 is a view illustrating the arrangement of the imaging sensor 107 and the pressing sensor 108 in the first embodiment.

FIG. 4 is a block diagram illustrating a configuration of an information processing apparatus 200.

FIG. 5 is a flowchart illustrating registration processing.

FIG. 6 is an explanatory view of a registration database.

FIG. 7 is as explanatory view of the registration database.

FIG. 8 is a flowchart illustrating authentication processing.

FIG. 9 is a view illustrating a first example of a user interface (UI).

FIG. 10 is a view illustrating a second example of the UI.

FIG. 11 is a view illustrating a third example of the UI.

FIG. 12 is a view illustrating a fourth example of the UI.

FIG. 13 is a view illustrating a fifth example of the UI.

FIG. 14 is a view illustrating a sixth example of the UI.

FIG. 15 is a view illustrating arrangement of an imaging sensor 107 and a pressing sensor 108 in a second embodiment.

FIG. 16 is a view illustrating the arrangement of the imaging sensor 107 and the pressing sensor 108 in the second embodiment.

FIG. 17 is a view illustrating arrangement of an imaging sensor 107 and a pressing sensor 108 in a third embodiment.

FIG. 18 is a view illustrating the arrangement of the imaging sensor 107 and the pressing sensor 108 in the third embodiment.

FIG. 19 is a view illustrating the arrangement of the imaging sensor 107 and the pressing sensor 108 in the third embodiment.

FIG. 20 is an explanatory view of a center of gravity position of contact.

### MODE FOR CARRYING OUT THE INVENTION

Hereinafter, embodiments of the present technology will be described with reference to the drawings. Note that description will be given in the following order.

<1. First Embodiment>

[1-1. Configuration of Device 100]

[1-2. Configuration of Information Processing Apparatus 200]

[1-3. Processing in Information Processing Apparatus 200]

[1-3-1. Registration Processing]

[1-3-2. Authentication Processing]

[1-3-3. UI Processing]

<2. Second Embodiment>

<3. Third Embodiment>

<4. Modifications>

<1. First Embodiment>

[1-1. Configuration of Device 100]

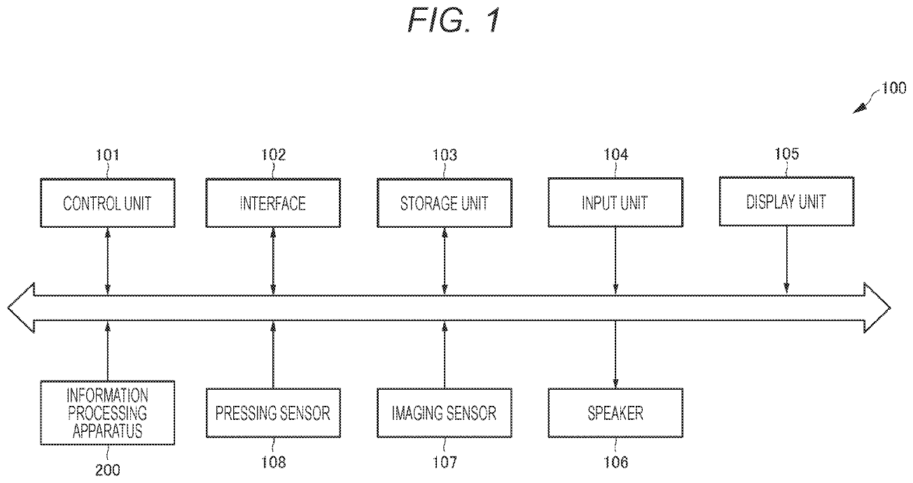

First, referring to FIG. 1, a configuration of a device 100 will be described. The device 100 includes a control unit 101, an interface 102, a storage unit 103, an input unit 104, a display unit 105, a speaker 106, an imaging sensor 107, a pressing sensor 108, and an information processing apparatus 200.

The control unit 101 includes a central processing unit (CPU), a random access memory (RAM), a read only memory (ROM), and the like. The CPU controls the entirety and each unit of the device 100 by executing various kinds of processing according to a program stored in the ROM and. issuing commands.

The interface 102 is an interface with other devices, the Internet, and the like. The interface 102 may include a wired or wireless communication interface. Furthermore, more specifically, the wired or wireless communication. interface may include cellular communication such as 3TTE, Wi-Fi, Bluetooth (registered trademark), near field communication (NFC), Ethernet (registered trademark), a High-Definition Multimedia Interface (HDMI) (registered trademark), a universal serial bus (USB), and the like. Furthermore, in a case where at least a part of the device 100 and the information processing apparatus 200 is achieved. by the same apparatus, the interface 102 can include a bus in the apparatus, data reference in a program module, and the like (hereinafter, the bus in the apparatus, the data reference in the program. module, and the like are also referred to as interfaces in the apparatus).

Furthermore, in a case where the device 100 and the information processing apparatus 200 are achieved in a manner distributed to a plurality of apparatuses, the interface 102 may include different kinds of interfaces for the respective apparatuses. For example, the interface 102 may include both the communication interface and the interfaces in the apparatus.

The storage unit 103 is, for example, a large capacity storage medium such as a hard disk or a flash memory. The storage unit 103 stores various kinds of applications, data, and the like used by the device 100. Furthermore, in a case where the information processing apparatus 200 operates on the device 100, a fingerprint image generated by the imaging sensor 107, a registration database, and the like are also stored in the storage unit 103.

The input unit 104 is used by a user to input various kinds of instructions and the like to the device 100. When an input is made to the input unit 104 by the user, a control signal corresponding to the input. is generated and supplied to the control unit 101. Then, the control unit 101 performs various kinds of processing corresponding to the control signal. In addition to a physical button, the input unit. 104 includes a touch panel, voice input by voice recognition, gesture input by human body recognition, and the like.

The display unit 105 is a display or the like that displays an image/video, a graphical user interface (GUI) for fingerprint image generation, and the like.

The speaker 106 outputs audio of content, audio for a user interface, and the like.

The imaging sensor 107 includes an imaging element (charge coupled device (CCD), complementary metal oxide semiconductor (CMOS), or the like) that photoelectrically converts incident light into a charge amount and outputs an imaging signal, an image signal processing unit that generates image data by subjecting the imaging signal to decode processing and performing analog/digital (A/D) conversion and the like. A fingerprint image is generated by the imaging sensor 107, and registration processing and authentication processing by the information processing apparatus 200 are performed using the fingerprint image.

The imaging sensor 107 irradiates a finger with light from a light source using the fact that a light reflection manner is different between a convex portion and a concave portion of the fingerprint, receives shade and shadow of incident light reflected by the finger at the imaging element, and then generates a fingerprint image.

In the present embodiment, description will be given assuming that a part of a living body is a finger of the user, an image of a fingerprint of the finger is generated. by imaging sensor 107, and authentication is performed using the fingerprint image.

The pressing sensor 108 detects that the user has brought the finger into contact with a contact surface T in order to generate a fingerprint image, and detects, as contact information, the degree of pressing (pressing force) against the contact surface T at the time of contact. In a first embodiment, a strain gauge is used as the pressing sensor 108. However, the pressing sensor 108 is not limited to a strain gauge, and may be an electrostatic capacitance sensor as described later or may be any other sensor such as a metal gauge, a semiconductor gauge, or a load cell as long as the sensor can detect the degree of pressing. In the present technology, the pressing sensor 108 that detects the degree of pressing of the finger is arranged around the imaging sensor 107, and the degree of pressing detected by the pressing sensor 108 is used as the contact. information to improve the accuracy and speed of biometric authentication.

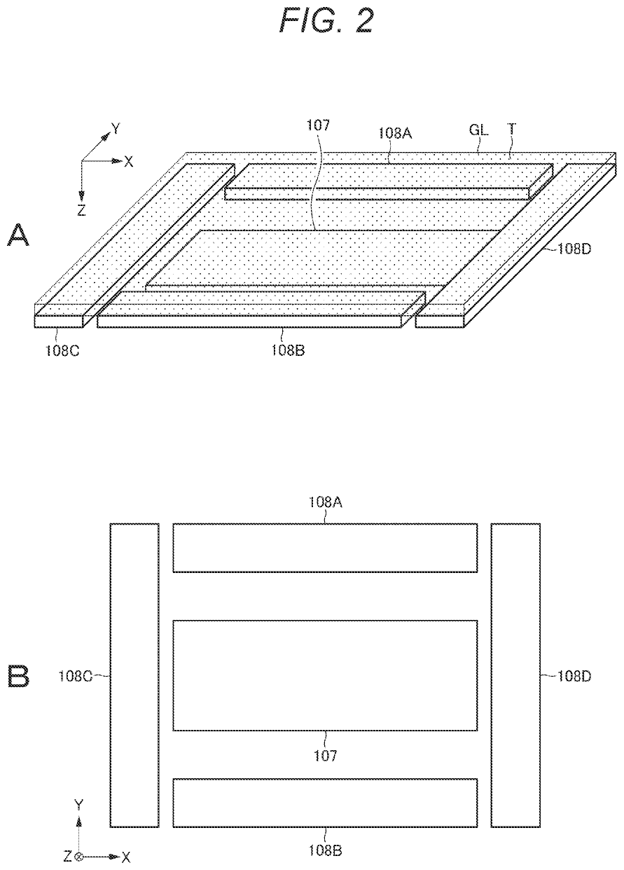

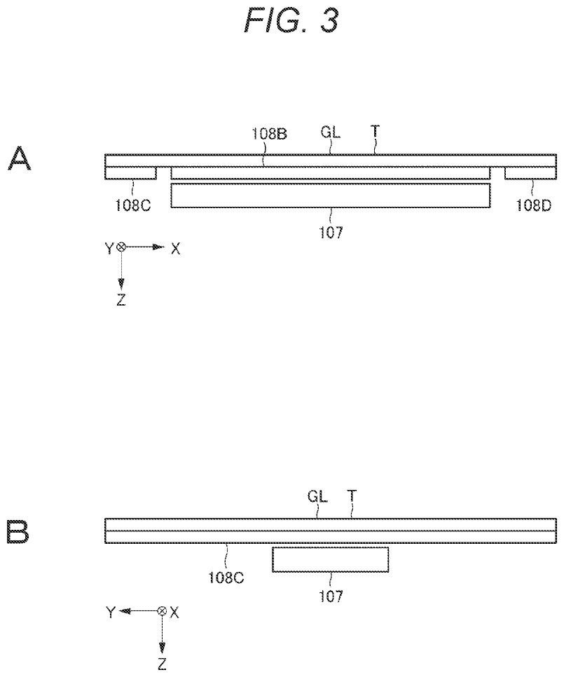

Here, referring to FIGS. 2 and 3, an arrangement configuration. of the contact surface T, the imaging sensor 107, and the pressing sensor 108 will be described. Note that in FIGS. 2 and 3, description will be given assuming that an X-axis direction is a width direction, a Y-axis direction is a vertical direction, and. a Z-axis direction is a downward direction (direction in which the user brings the finger into contact).

As illustrated In FIGS. 2A, 3A, and 3B, a glass plate GL whose upper surface is the contact surface T, a strain gauge 108A, a strain gauge 108B, a strain gauge 108C, and a strain gauge 108D as the pressing sensor 108, and the imaging sensor 107 are arranged in this order from the top so as to form a hierarchical structure. Note that in FIG. 2B, the glass plate GE is omitted.

As illustrated in a plan view of FIG. 2B, the four strain gauges 108A, 108B, 108C, and 108D are arranged around the imaging sensor 107 so as to surround. the imaging sensor 107.

The strain gauge 108A and the strain Gauge 108B that oppose each. other can detect a. load position in the Y direction. Furthermore, the strain gauge 108C and the strain gauge 108D that oppose each other can detect a load position in the X direction. Moreover, the degree of pressing in the Z direction can be calculated using the sum of values of the pressing forces detected by the strain gauges 108A to 108D. In the present technology, the degree of pressing is calculated as the pressing force.

If the pressing sensor 108 detects that the user has brought the finger into contact with the contact surface T, the imaging sensor 107 generates a fingerprint image. Furthermore, if the pressing sensor 108 is pressed by the contact of the finger with the contact surface T, the degree of pressing as information associated with information regarding the contact of the finger (contact information) is acquired. Since the degree of pressing by the finger only needs to be indirectly detected in this manner, the glass plate GL may be disposed on the pressing sensor 108. Note that since the degree of pressing only needs to be indirectly detected, the pressing sensor 108 may be disposed. below the imaging sensor 107.

The device 100 may be any device such as a wearable device of a wristwatch type, a glasses type, a wristband type, a ring type, or the like in addition to a smartphone, a personal computer, a card type device, an Internet of Things (IoT) device, a security system, and a door lock system of a house, a car, or the like as long as the device can be equipped with a biometric authentication function using a fingerprint.

Note that the interface 102, the input unit 104, the display unit 105, and the speaker 106 are not essential configurations. In a case where the registration and authentication of the biometric information are completed only by the device 100 and the information processing apparatus 200 without requiring communication with the outside, a function of communication with the outside in the interface is unnecessary. Furthermore, in a case where an instruction or information is presented to the user only by display on the display unit 105, the speaker 106 is unnecessary, and conversely, in a case where an instruction or information is presented to the user only by audio, the display unit 105 is unnecessary.

[1-2. Configuration of Information Processing Apparatus 200]

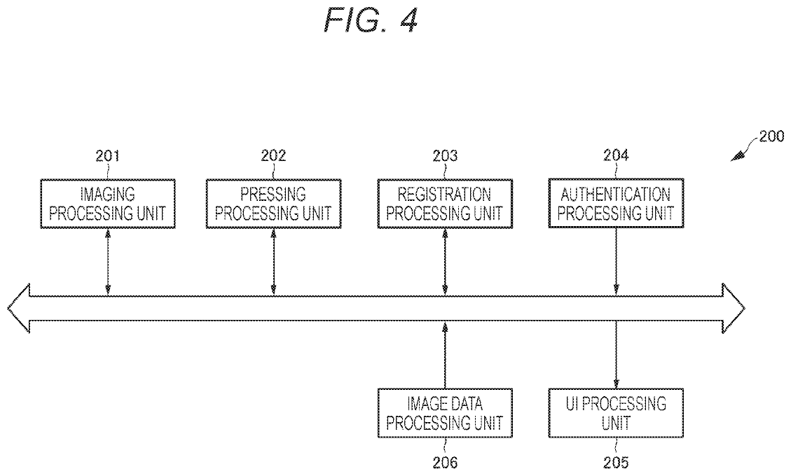

Next, referring to FIG. 4, a configuration of the information processing apparatus 200 will be described. The information. processing apparatus 200 includes an imaging processing unit 201, a pressing processing unit 202, a registration processing unit 203, an authentication processing unit 204, a user interface (UI) processing unit 205, and an image data processing unit 206.

Processing in the present technology is divided into two stages: a registration stage of the fingerprint image and as authentication stage using the fingerprint image. The contact of the finger with the contact surface T for the registration processing is contact for registration in the claims. Furthermore, the fingerprint image that is an image of the part of the living body of the user, the image being generated by the contact for registration, the image being used for registration, is an image for registration in the claims. Moreover, the contact information in the contact for registration is contact information at the time of registration in the claims.

Furthermore, the contact of the finger with the contact surface T for the authentication processing after the registration processing is completed is contact for authentication in the claims. Furthermore, the fingerprint image generated by the contact for authentication and used for authentication is an image for authentication in the claims. Moreover, the contact. information in the contact for authentication is contact information at the time of authentication in the claims.

If the pressing sensor 108 detects the contact. of the finger with the contact surface T, the imaging processing unit 201 controls the operation of the imaging sensor 107 to cause the imaging sensor 107 to generate a fingerprint image. Furthermore, the imaging processing unit 201 supplies the fingerprint image to the registration processing unit 203 or the authentication processing unit 204 according to whether the contact of the finger with the contact surface T by the user is for registration purpose or for authentication purpose.

The pressing processing unit 202 acquires the contact information from the pressing sensor 108, and supplies the contact information. to the registration processing unit 203 or the authentication processing unit 204 according to whether the contact of the finger with the contact surface T by the user is for registration purpose or for authentication purpose. Furthermore, the pressing processing unit 202 supplies the contact. information to the UI processing unit. 205 as necessary for the UI processing.

Note that whether the contact of the finger with the contact surface T by the user is for registration purpose or for authentication purpose can be checked by referring to, for example, a state of the device 100. In a state in which the setting screen of the device 100 or an application requests the registration of the fingerprint image, the contact of the finger with the contact surface T by the user is for registration purpose. Furthermore, in a case where the device 100 requests fingerprint authentication for activation or use of an application, the contact of the finger with the contact surface T by the user is for authentication purpose.

The registration processing an it 203 performs, as living body-related processing, registration of a fingerprint image for registration as biometric information for authentication. The registered fingerprint image for registration is stored in, for example, a registration database included in the storage unit 103.

The authentication processing unit 204 performs, as the living body-related processing, biometric authentication processing using the fingerprint by comparing a fingerprint image for authentication with the fingerprint image for registration in the registration database.

The authentication processing unit 204 performs the biometric authentication processing on the basis of feature. information. The, feature information is a generic term for information used in authentication using a finger, and includes various kinds of information that can be acquired from a fingerprint image. Examples of the feature information include an. overall shape of a. ridge and a break, branch point, end point, and the like of the ridge that are characteristic configurations of the ridge. Furthermore, a position of a sweat gland, the torture of the finger, and the like may be included. The accuracy of authentication can be improved by using more information. Although details will be described later, in the present technology, the degree of pressing the finger against the contact surface T, a contact position of the finger, and the like can also be included in the feature information.

The authentication processing unit 204 performs fingerprint authentication by comparing feature information that can be acquired from the fingerprint image for registration registered in the registration database with feature information that can be acquired from the fingerprint image for authentication and then by detecting a matching point by a publicly known fingerprint authentication method.

The UI processing unit 205 performs UI selection for prompting the user to bring a finger into contact with the contact surface T in order to generate a fingerprint image, UI display processing on the display unit 105, and the like.

The image data processing unit 206 performs processing of checking whether the captured fingerprint image is of a quality to be usable as the biometric information for authentication and the like.

The information processing apparatus 200 may be achieved by executing a program, and the program may be installed in the device 100 in advance or may be distributed by downloading or in a form of a storage medium or the like and installed by a manufacturer, a company, a user, or the like. Furthermore, the information processing apparatus 200 may operate in an external device different from a device, for example, a server, a cloud, or the like. Moreover, the information processing apparatus 200 may be achieved not only by the program but also by combining a dedicated device, a circuit, and the like by hardware having a function of the program.

[1-3. Processing in Information Processing Apparatus 200]

[1-3-1. Registration Processing]

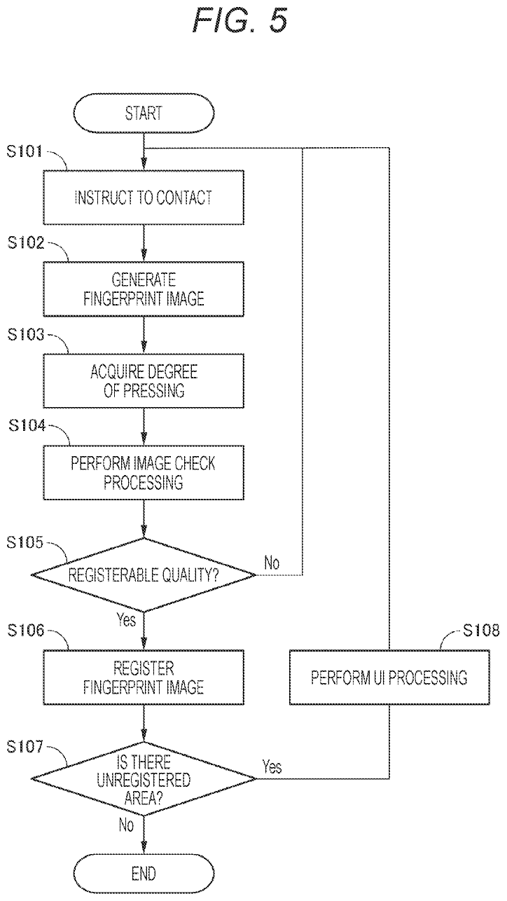

Next, processing in the information processing apparatus 200 be described. First, referring to a flowchart of FIG. 5, the registration processing will be described. The registration processing is processing in which the fingerprint image for registration of the user that is biometric information is registered in order to use the fingerprint image for registration for the fingerprint authentication.

First, in step S101, the user is instructed to bring a finger into contact with the contact surface T in order to generate a fingerprint image for registration. This instruction can be performed, for example, by outputting audio from the speaker 106 that displays a message on the display unit 105 of the device 100.

If the user brings a finger into contact with the contact surface T, the imaging sensor 107 generates a fingerprint image for registration in step 8102. Furthermore, in step S103, the pressing sensor 108 acquires the degree of pressing by the user against the contact surface T as the contact information at the time of registration. Note that for the convenience of the flowchart, step S102 is the fingerprint image generation, and step S103 is the acquisition of the degree of pressing, but in practice, step S102 and step S103 are performed substantially simultaneously in parallel.

Next, in step S104, the image data processing unit 206 performs image check processing. The image check processing is to check whether the fingerprint image for registration is of a quality to be usable for registration. For example, in a case where the brightness of the image is detected and the brightness a first predetermined value or less, it is determined that the image is too dark and the fingerprint image is not of a quality to be usable for registration. Furthermore, in a case where the brightness is a second predetermined value or more for a reason such as that too much external light enters, it is determined that the image is too bright and the fingerprint image is not of a quality to be usable for registration. Furthermore, even in a case where an unclear portion is detected in a part of the fingerprint image for registration, it is determined that the fingerprint image for registration is not of a quality to be usable for registration.

In a case where the fingerprint image for registration is of a quality to be usable for registration, the processing proceeds from step S105 to step S106 (Yes in step S105). Meanwhile, in a case where the fingerprint image for registration is not of a quality to be usable for registration, the processing returns to step S101, and the user is instructed again to bring the finger into contact with the contact surface T in order to generate a fingerprint image for registration.

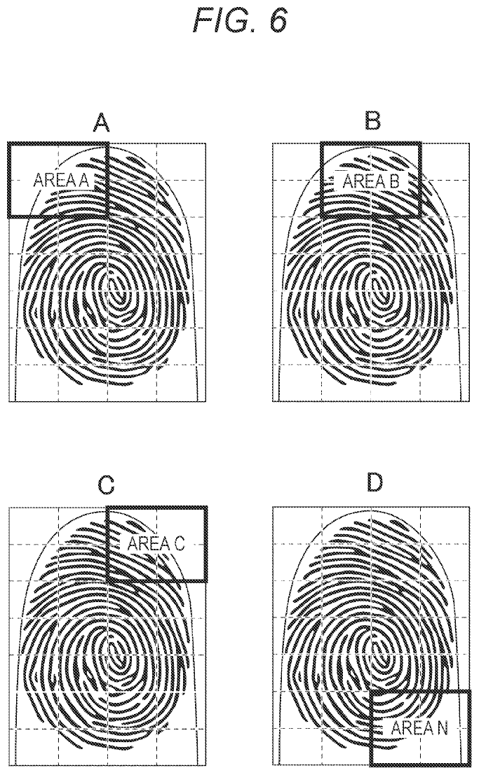

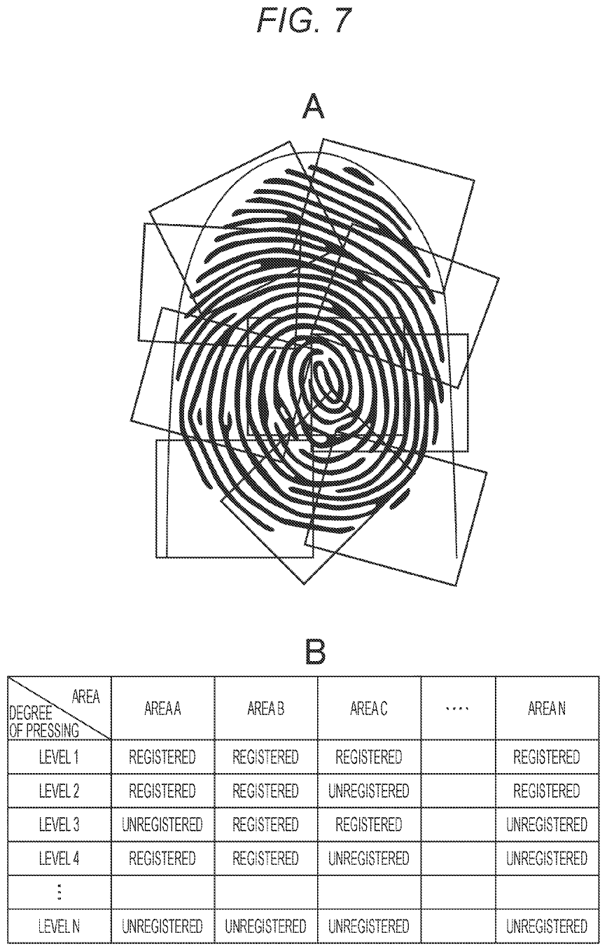

Next, in step S106, the registration processing unit 203 registers the fingerprint image for registration in the registration database. As illustrated in areas A, B, C, and N in FIGS. 6A to 6D, in the registration database, the user's finger is divided into a plurality of regions in advance, an overlapping region in consideration of a positional shift of the contact of the finger is provided, and the overlapping region is divided into a plurality of areas. In FIG. 6, as an example, four regions that are 2×2 regions constitute one area, and two regions are overlapped with each other between adjacent areas. The reason why the overlap region is provided in this manner is that the user does not necessarily accurately bring the finger into contact with a position indicated to the user by the UI and it is difficult to accurately make the user bring the finger into contact with the position through guidance by the UI. As illustrated by rectangular frames on a fingerprint in FIG. 7A, a fingerprint image for registration obtained by actual imaging has a positional deviation with respect to the area, but it is sufficient if a fingerprint image of a target area can be acquired finally in an exhaustive manner without omission.

As illustrated in FIG. 7B, there is adopted a configuration in which the fingerprint image for registration is registered for each area while corresponding to a level of the degree of pressing by the user against the contact surface T. As described above, the registration processing is processing in which a plurality of images for registration generated by a plurality of times of contact for registration with the contact surface T is registered according to the level of the degree of pressing and the fingerprint images for registration is registered in all the areas. Therefore, the plurality of images for registration registered while corresponding to the level of the degree of pressing in the registration database is images of different regions of the fingerprint of the finger that is the part of the living body.

Since a value of a number N that represents the level of the degree of pressing depends on the resolution of the pressing sensor 108, the value of the number N is considered to be experimentally obtained. In a case where the pressing sensor 108 is a pressure gauge, pressure is used, and in a case where the pressing sensor 108 is of an electrostatic capacity type, an area of contact is used.

A boundary of the levels may be set to a predetermined number in advance according to the resolution of the pressing sensor 108. Furthermore, after the maximum value and the minimum value of the degree of pressing by the user are measured, the number of levels may be set according to a range of the degrees of pressing. For example, in a case where the range of the degrees of pressing is large, the boundary is set so that the number of levels increases. Furthermore, in a case where the range of the degrees of pressing is small, the boundary is set so that the number of levels increases. Furthermore, clustering can be performed on the basis of the inner product of feature vectors to classify the levels.

Since the degree of pressing by the user is unknown until actual contact is made, a plurality of provisional levels may be set in advance, and after the pressing sensor 108 actually detects a plurality of degrees of pressing, the levels may be reset according to the degrees of pressing. For example, in a case where five levels that are levels 1 to 5 are set in advance and as a result of detecting a plurality of degrees of pressing by the pressing sensor 108 and pressing at level 4 or higher has not been performed, level 4 or higher is unnecessary and the levels are reset to only 1 to 3. Alternatively, in a case where the number of times of pressing with the degrees of pressing of level 4 and level 5 is extremely small compared to levels 1 to 3, it is also possible to reset level 4 and level 5 so that level 4 and level 5 are integrated into one level.

Since the finger is deformed according to the degree of pressing against the contact surface T or the ridge of the fingerprint is deformed, more accurate authentication can be achieved by hierarchizing and registering fingerprint images for each degree of pressing according to the degree of pressing by the user.

Next, in step S107, the registration processing unit 203 checks whether or not there is an area in which a fingerprint image for registration is not registered in the registration database. In a case where the fingerprint images for registration are registered while corresponding to all the areas in all the degrees of pressing, the processing ends (No in step S107).

Meanwhile, in a case where there is an area in which a fingerprint image for registration is not registered, the processing proceeds to step S108 (Yes in step S107).

Then, in step 3108, the UI processing unit 205 performs UI selection, and the display unit 105 performs UI display processing. The processing by the UI processing unit 205 is a UI that checks the registration database and prompts the user to bring the finger into contact so that the user generates a fingerprint image of the area whose fingerprint image is not registered yet. A specific example of the UI will be described later.

The registration processing is performed as described above. If the registration processing is completed, all of the fingerprint image in the level of each degree of pressing and the fingerprint image of each area have been registered in the registration database. If the fingerprint images of all the areas are registered, the fingerprint image of the entire user's finger has been registered for each level of the degree of pressing.

Note that the level of the degree of pressing may be set in advance, and the registration processing may be performed until the fingerprint images of all the areas at all the levels of the degree of pressing are registered. In a case where there is a bias in the degree of pressing by the user, the fingerprint images at all the levels does not need to be registered.

For example, as a result of a plurality of times of contact with the contact surface T, in a case where the fingerprint images of all the areas in level 1 to level 3 have been registered, but the degree of pressing by the user is weak, and the fingerprint images of the areas in level 4 or higher are not registered, or the number of registered fingerprint images is a predetermined number or less, the registration database may exclude level 4 or higher.

Furthermore, in a case where the number of registrations of fingerprint images of the areas in a plurality of levels that are level 4 or higher is a predetermined number or less, the plurality of levels may be integrated into one level. Note that the term “level 1 to level 3” and the term “level 4 or higher” are merely examples for convenience or description, and the present technology is not limited to those levels.

Furthermore, in a case where even if the fingerprint image is generated a predetermined number of times, there is a bias in the level of the degree of pressing and/or the area and the fingerprint images of all the areas all the levels of the degree of pressing cannot be registered, the bias and distribution can also be used for the authentication processing as the contact information.

Note that a plurality of adjacent areas may be combined into one area, and a plurality of fingerprint images may be joined together by stitching and registered as fingerprint images of the combined area.

[1-3-2. Authentication Processing]

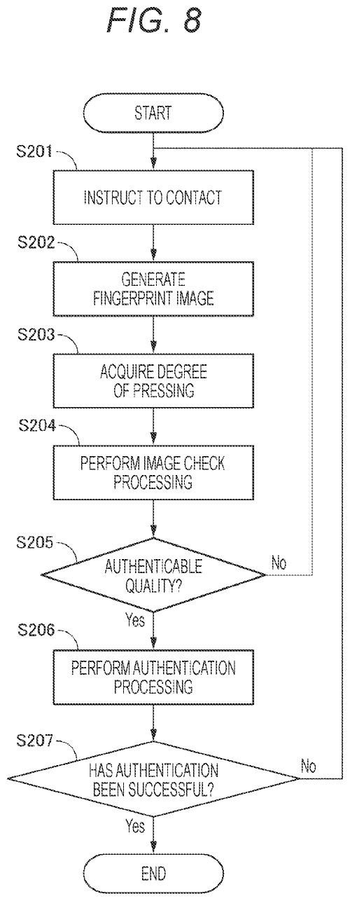

Next, referring to a flowchart of FIG. 8, the authentication processing will be described. This authentication processing is processing in which it is checked whether the finger that has been in contact with the contact surface T is a finger whose fingerprint has been registered, using the fingerprint image for registration registered in the registration database and the fingerprint image for authentication generated for authentication.

Steps S201 to S204 are the same as steps S101 to S104 in the registration processing, and thus the description thereof will be omitted.

In step S205, in a case where the fingerprint image for authentication is of a quality to be usable for the authentication processing, the processing proceeds to step S206 (Yes in step S205). Meanwhile, in a case where the fingerprint image for authentication is not of a quality to be usable for the authentication processing, the processing returns to step S201, and the user is instructed again to bring the finger into contact with the contact surface T in order to generate a fingerprint image for authentication (No in step S205).

In a case where the fingerprint image for authentication is of a quality to be usable for the authentication processing, the authentication processing unit 204 performs the authentication processing in step S206. Note that in the registration processing, the contact and the generation of the fingerprint image have been repeatedly performed until the fingerprint image for registration is registered in all the areas, but in the authentication processing, the generation of the fingerprint image only needs to be performed once except for a case where the fingerprint image for authentication is not of a quality to be usable for the authentication processing.

In the authentication processing, processing is performed using the fingerprint image for registration, the contact information at the time of registration, the fingerprint image for authentication, and the contact information at the time of authentication. In the authentication processing, processing is performed with reference to the fingerprint image for registration. registered while corresponding to the same level of the degree of pressing (contact information at the time of registration) as a level of the degree of pressing the finger against the contact surface T at the time of the contact for authentication (contact information at the time of authentication).

For example, in a case where the degree of pressing the finger against the contact surface T at the time of the contact for authentication is level 2, the authentication processing is performed with reference to the fingerprint image for registration registered while corresponding to the degree of pressing of level 2 in the registration database. Then, the feature information that can be acquired from the fingerprint image for authentication is compared with the feature information that can be acquired from all the corresponding fingerprint images for registration of level 2, and the biometric authentication processing is performed according to whether or not the feature information that can be acquired from the fingerprint image for authentication matches the feature information that can be acquired from all the corresponding fingerprint images for registration of level 2.

In the fingerprint, the shape of the ridge changes according to the degree of pressing of the finger, but the accuracy of the fingerprint authentication can be improved by using the degree of pressing as the contact information for the authentication.

Note that in a case where the authentication has not been successful with the fingerprint image for registration registered while corresponding to the same level of the degree of pressing as a level of the degree of pressing at the time of the contact for authentication, the authentication processing may be performed with the fingerprint image for registration corresponding to a level of the degree of pressing that is a nearby level. For example, in a case where the degree of pressing of the finger in the contact for authentication is level 2 and the authentication has not been successful with reference to the fingerprint image for registration registered while corresponding to the degree of pressing of level 2 in the registration database, the authentication processing is performed with the fingerprint image for registration in level 1 or level 3 that is a nearby degree of pressing. As a result, the authentication processing can be efficiently performed even if the degree of pressing is different.

In a case where the authentication has been successful on the basis of a determination that the fingerprint image for authentication matches the fingerprint image for registration registered in the registration database in the authentication processing, the process in ends (Yes in step S207). Meanwhile, the processing in which the authentication has failed returns from step S207 to step S201, and the user is instructed again to bring the finger into contact with the contact surface T for imaging for the fingerprint registration (No in step S207).

Note that it is assumed that a false finger is different in flexibility from a real finger, and even in a case where pieces of the feature information of the fingerprint are very similar to each other, when the degrees of pressing are different from each other, it is also possible to determine that the finger is a false finger and then determine that the authentication has failed.

[1-3-3. UI Processing]

Next, the UI displayed on the display unit 105 by the UT processing unit 205 will be described. The UI is for providing information to the user and guiding the user to bring the finger into contact with the finger a plurality of times in various modes and at various degrees of pressing in order to register fingerprint images for registration of the plurality of regions of the finger in the registration database.

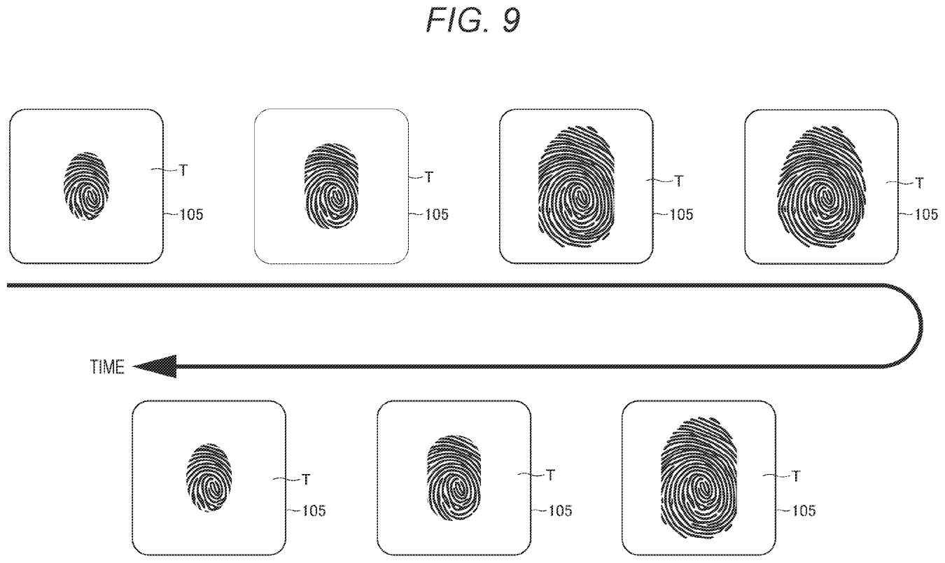

FIG. 9 is a first example of the UI. In the first example, in a case where the display unit 105 also serves as the contact surface T, an image indicating a fingerprint for prompting to bring the finger into contact with the display unit 105 is displayed. The image of the fingerprint is changed so as to indicate a state in which a shape changes according to the degree of pressing over time. The user changes the degree of pressing according to a change in the display and bring the finger into contact, whereby the imaging sensor 107 can efficiently generate a plurality of fingerprint images at the degree of pressing.

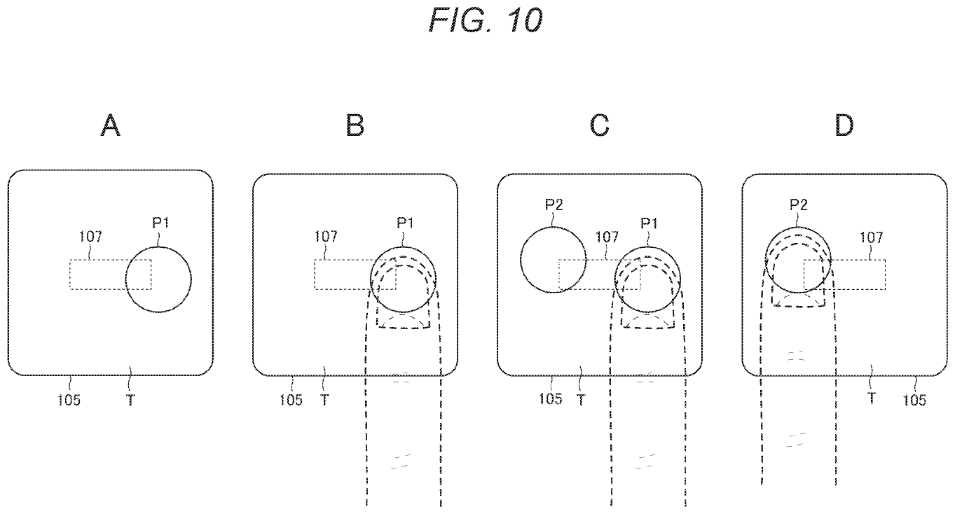

FIG. 10 illustrates a second example of the UI. In the second example, in a case where the display unit 105 also serves as the contact surface T, an icon indicating a position on the contact surface T with which the finger is to be brought into contact is displayed. In a case where the imaging sensor 107 is smaller than the finger, a fingerprint image of the entire finger cannot be generated by single contact. Therefore, a position of the icon is changed and displayed so that a position of the finger that overlaps on the imaging sensor 107 differs each time the contact is made.

In the example of FIG. 10, an icon P1 is first displayed as illustrated in FIG. 10A, and if the user brings the finger into contact with the icon 21 as illustrated in FIG. 10B, an icon P2 is next displayed at a position different from a position of the icon P1 as illustrated in FIG. 10C. If the user brings the finger into contact with the icon P2, the next icon is displayed at a position different from a position of the icon P2.

This is repeated until fingerprint images for registration of all the areas of the finger are generated. The user brings the finger into contact with a position where an. icon is displayed so that the user follows the icon, whereby a fingerprint image for registration of the entire finger can be generated. Note that in a case where the user brings the finger into contact with the position indicated by the icon, an icon indicating a position at which the finger is to be brought into contact next may be displayed at a position not hidden by the finger.

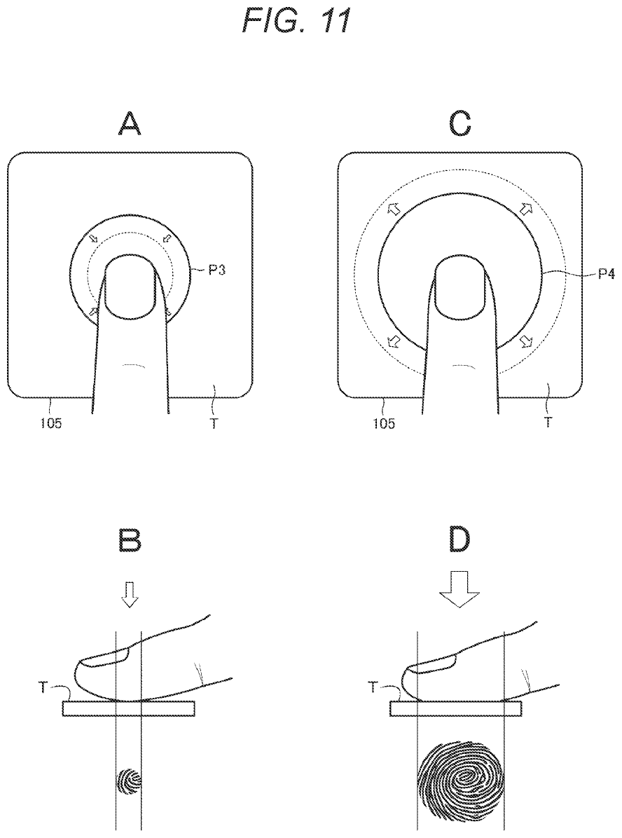

FIG. 11 illustrates a third example of the UI. In the third example, in a case where the display unit 105 also serves as the contact surface T, an icon P3 for instructing pressing of the finger against the contact surface l is displayed. For example, as illustrated in FIG. 11A, in a case where it is instructed to contact at a weak degree of pressing (pressing force of a predetermined value or less), a small circle, inward arrows, a dotted line indicating a guidance destination of the arrows, and the like are displayed as the icon P3. As a result, as illustrated in FIG. 11B, the user can be guided to contact at the weak degree of pressing by the small circle, the inward arrows, and the dotted line. Furthermore, in a case of guiding to contact with strong pressing force (pressing force of a predetermined value or more), a large circle, outward arrows, a dotted circle, or the like are displayed as an icon P4 as illustrated in FIG. 11C. As a result, as illustrated in FIG. 11D, the user can be guided to contact at a strong degree of pressing.

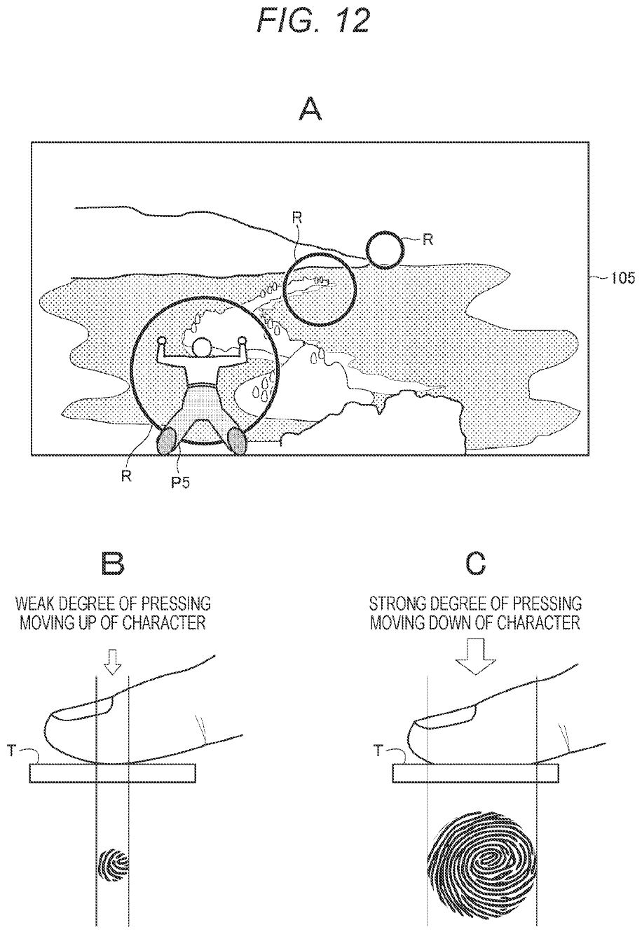

FIG. 12 illustrates a fourth example of the UI. In the fourth example, the user is guided to contact at a strong degree of pressing or a weak degree of pressing through a game that operates a character displayed on the display unit 105. For example, in a game in which a character P5 that is a doll swimming in the air is operated so as to pass through. a plurality of continuous virtual rings R, the character P5 moves up and down according to the degree of pressing the finger against the contact surface as illustrated in FIGS. 12B and 12C. By changing a position of the virtual ring R, the finger can be brought into contact with the contact surface T at various degrees of pressing. In this example, the display unit 105 may also serve as the contact surface or the display unit 105 and the contact surface T may be separate.

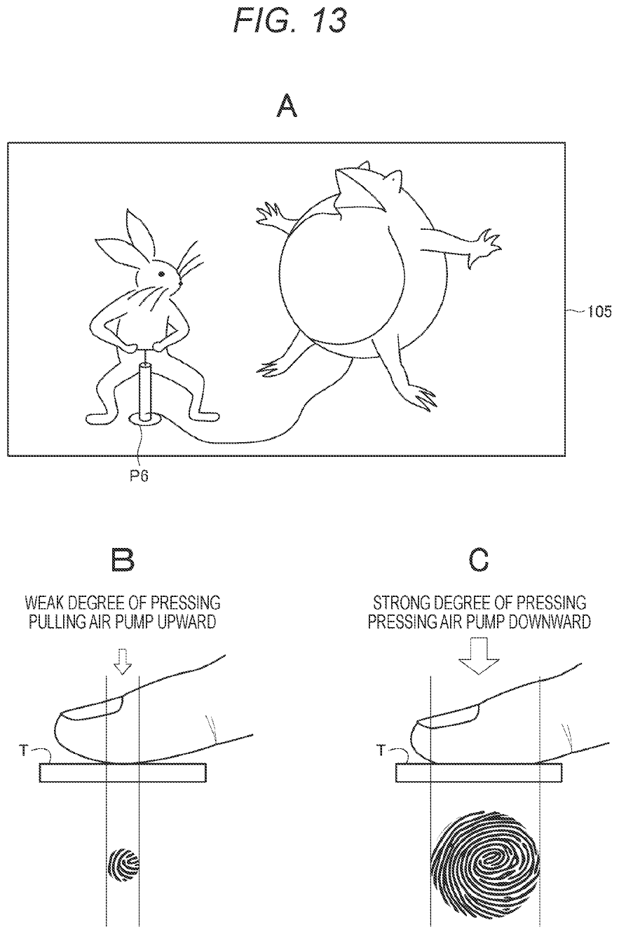

Furthermore, as illustrated in a fifth example of FIG. 13, as a game, there is also an example in which a character changes so that the degree of injecting a gas can be adjusted by making a change in the degree of pressing against the contact surface T correspond to the vertical operation of an air pump P6. In this example, as illustrated in FIGS. 13B and 13C, the vertical operation with respect to the air pump P6 changes according to the degree of pressing the finger against the contact surface T. Moreover, as a game that makes the user think of a vertical movement, there is also an example in which the degree of pressing is made to correspond to heart massage to a human character.

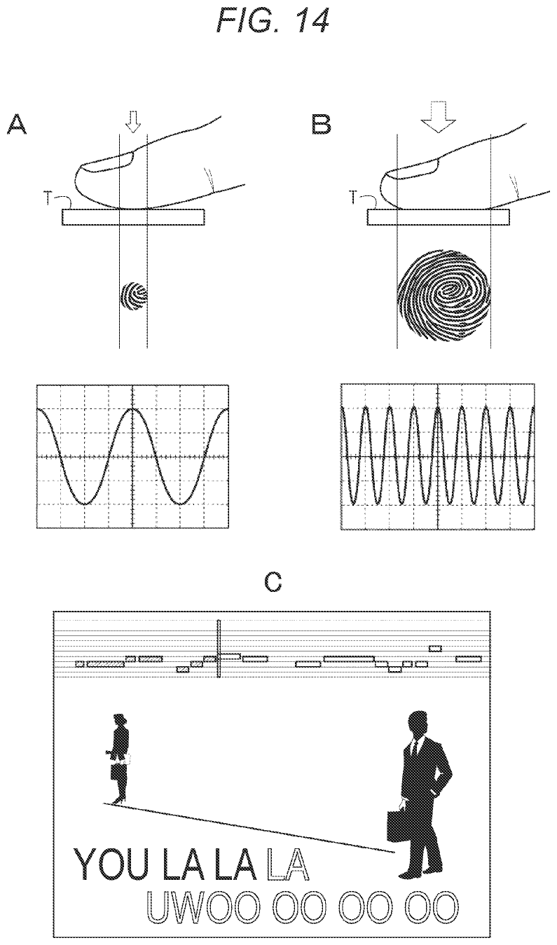

FIG. 14 illustrates a sixth example of the UI. In the sixth example, the degree of pressing the finger in contact against the, contact surface T is associated with a pitch of a sound output from the speaker 106. For example, as illustrated in FIG. 14A, a pitch of output sound is decreased as the degree of pressing becomes weaker, and as illustrated in FIG. 14B, a pitch of output sound is increased as the degree of pressing becomes stronger. As a result, the user can intuitively grasp the strength and weakness of the degree of pressing. By using this, for example, as illustrated in FIG. 14C, the user can be guided to bring the finger into contact with the contact surface T at various degrees of pressing by making a musical pitch of a song in karaoke correspond to the degree of pressing.

All the UIs described above can be achieved by the UI processing unit 205 acquiring information regarding the degree of pressing from the pressing processing unit 202 and performing display processing in association with the degree of pressing and a change in display (movement of a character, a change of an icon, and the like). For example, the finger is repeatedly guided to be slowly brought into contact with the contact surface T by the UIs described above and the finger is repeatedly guided in a direction away from the contact surface T by the is described above, whereby fingerprint images of a plurality of degrees of pressing can be generated by single contact. Furthermore, the finger is guided. to be brought into contact with an area not generated in order to complete the registration database, whereby the registration database can be efficiently completed.

<2. Second Embodiment>

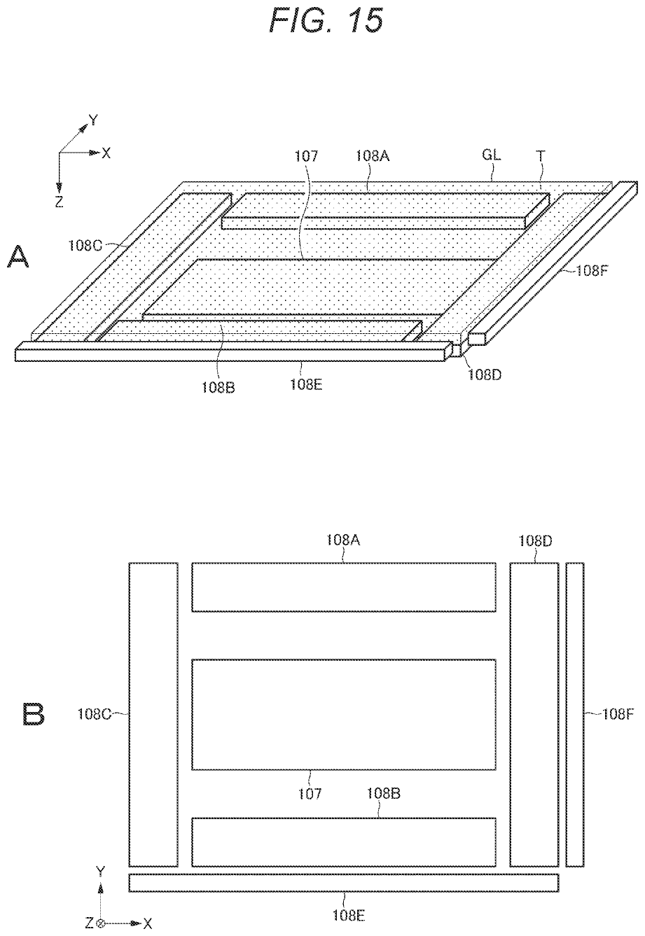

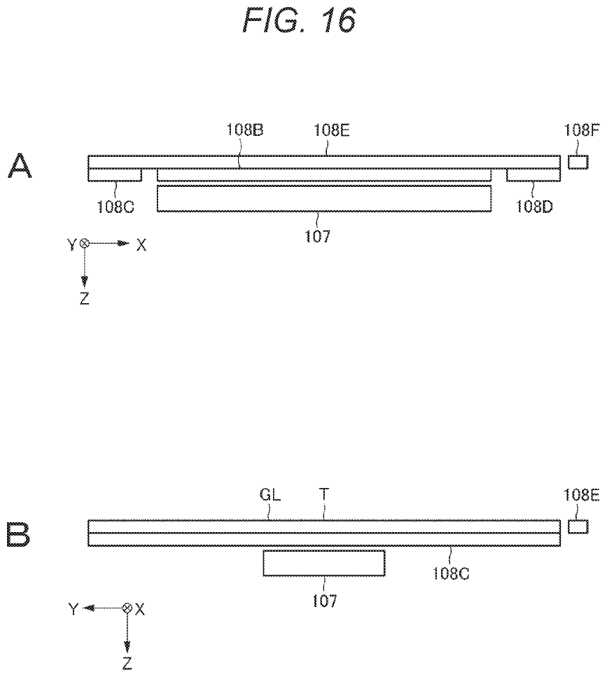

Next, referring to FIGS. 15 and 16, a second embodiment of the present technology will be described. The second embodiment is different from the first embodiment in. that the arrangement of a strain gauge as a pressing sensor 108 is different from that of the first embodiment. An imaging sensor 107 and strain gauges 108A to 108D are similar to those of the first embodiment. In the second embodiment, a strain gauge 108E and a strain gauge 108F as a pressing sensor 108 are further provided. Other configurations are similar to those of the first embodiment. Note that in FIG. 15B, a glass plate GL is omitted.

As illustrated in FIGS. 15 and 16, the strain gauge 108E is provided outside the strain gauge 108B in a plan view, and the strain gauge 108F is provided outside the strain gauge 108D. Moreover, the strain. gauge 108E and. the strain gauge 108F are provided so as to be positioned at the same height as the height of the glass plate GL as a contact surface T in a side view. The strain gauge 108E is for detecting shear force in an X direction. Furthermore, the strain gauge 108F is for detecting shear force in a Y direction.

If the user rotates the finger or shifts the finger while bringing the finger into contact with the contact surface T, the glass plate GL moves, the strain gauge 108F is pressed against the glass plate GL in the X direction, and the strain gauge 108E is pressed against the glass plate GL in the Y direction. The shear force can be detected by detecting pressing of the glass plate GL against the strain gauge 108E and the strain gauge 108E.

The shear force is also a characteristic of the contact by the user and can be used for authentication processing as one piece of the contact information. Furthermore, since as for contact that generates shear force, there is a possibility that the accuracy of the authentication processing may decrease due to deformation of the fingerprint, the user may be notified to change a method of bringing the finger into contact with the contact surface T in a case where the shear force is detected. Moreover, in a case where a device 100 has a vibration function and shear force is detected, the device 100 may be vibrated to forcibly eliminate the shear force.

Note that the strain gauge 108E may be provided. outside the strain gauge 108A, and the strain gauge 108F may be provided outside the strain gauge 108C. Moreover, a strain gauge for detecting the shear force may be provided in four directions so as to surround the strain gauge 108A, the strain gauge 108B, the strain gauge 108C, and the strain gauge 108D.

<3. Third Embodiment>

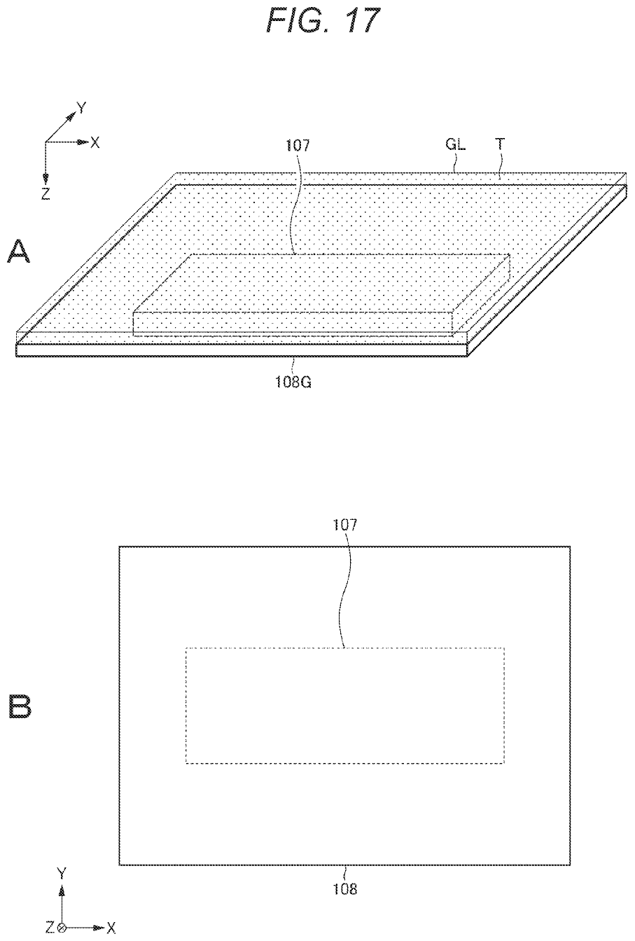

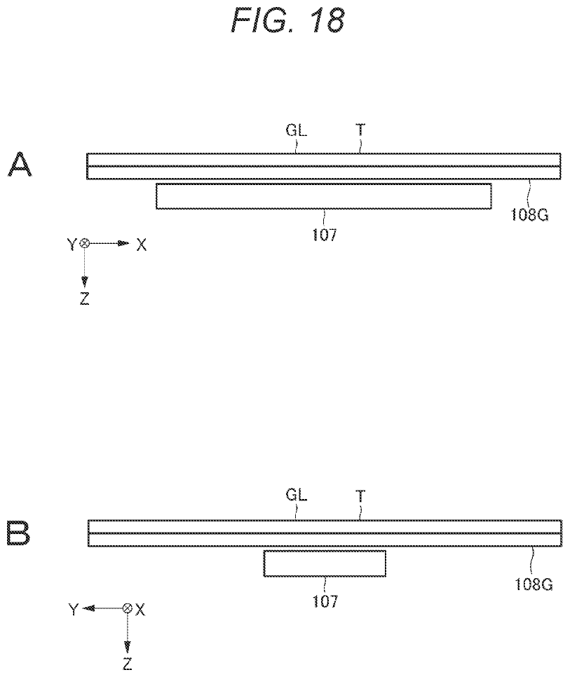

Next, referring to FIGS. 17 and 18, a third embodiment of the present technology will be described. The third embodiment is different from the first embodiment in that an electrostatic capacitance sensor is used as a pressing sensor 108 instead of a strain gauge. Other configurations are similar to those of the first embodiment. Note that in FIG. 17B, a glass plate GL is omitted.

The electrostatic capacitance sensor is a non-contact sensor that detects an object from a change in electrostatic capacitance generated between the electrostatic capacitance sensor and a human hand or the like. The electrostatic capacitance sensor can detect an area of contact of a finger in the contact surface T, a center of gravity position of contact of the finger, and the like.

FIGS. 17 and 18 illustrate a first arrangement example of the electrostatic capacitance sensor, and an electrostatic capacitance sensor 108G is provided so as to overlap above an imaging sensor 107 and below the glass plate GL. In this case, since a fingerprint image is generated by the imaging sensor 107 below the electrostatic capacitance sensor 108G, the electrostatic capacitance sensor 108G needs to be transparent.

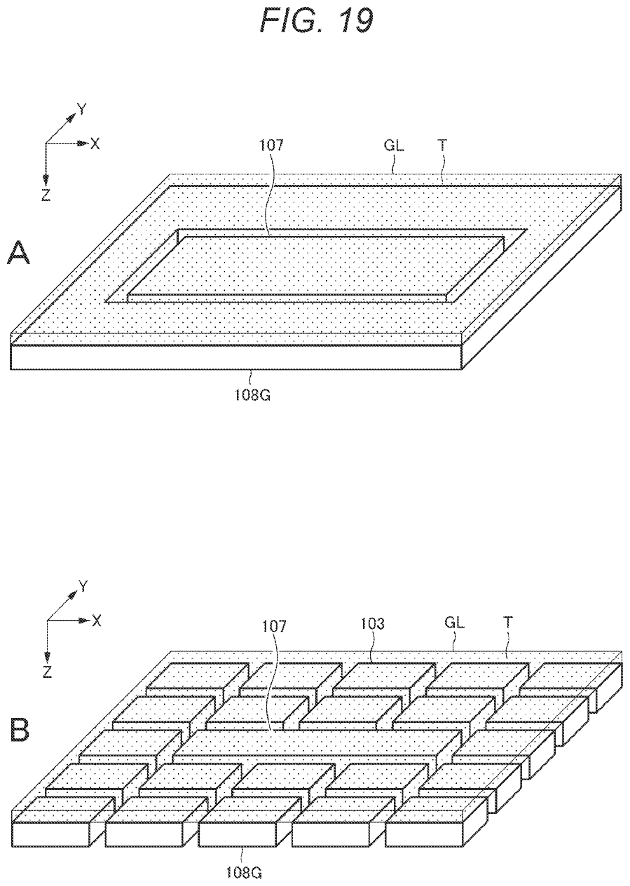

FIG. 19A illustrates a second arrangement example of the electrostatic capacitance sensor, and the electrostatic capacitance sensor 108G is provided so as to surround the imaging sensor 107. In this case, since the imaging sensor 107 and the electrostatic capacitance sensor 108G do not overlap each other, the electrostatic capacitance sensor 108G does not need to be transparent.

FIG. 19B illustrates a, third arrangement example of the electrostatic capacitance sensor, and a plurality of cellular electrostatic capacitance sensors 108G is provided so as to surround the imaging sensor 107. In this case, since the imaging sensor 107 and the electrostatic capacitance sensor 108G do not overlap each other, the electrostatic capacitance sensor 108G does not need to be transparent.

The electrostatic capacitance sensor 108G can detect, as the contact information, the area of contact and the center of gravity position of the contact of the finger in the contact surface T, and use the area of contact and the center of gravity position of contact of the finger for biometric authentication processing. Therefore, the electrostatic capacitance sensor 108G needs to have a size capable of including the entire finger in contact with the contact surface T.

In a case where the electrostatic capacitance sensor 108G detects the area of contact of the finger, a pressing processing unit 202 can estimate the degree of pressing from the area of contact. If the area of contact of the finger increases, detected capacitance increases, so that the degree of pressing the finger against the contact surface T can be calculated. The larger the area of contact of the finger, the larger the degree of pressing, and the smaller the area of contact, the smaller the degree of pressing.

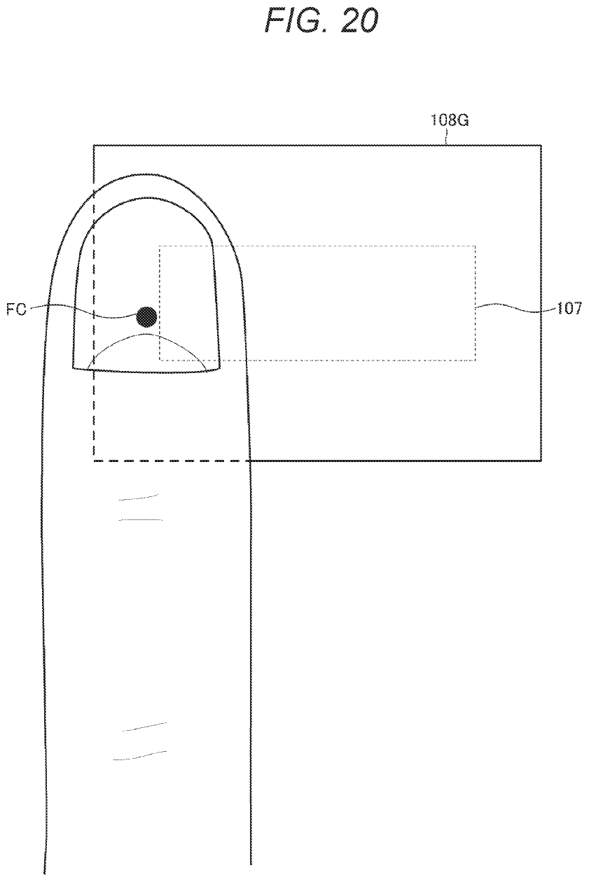

Furthermore, as illustrated in FIG. 20, a center of gravity position FC of the contact of the finger is detected by the electrostatic capacitance sensor 108G, whereby it is possible to estimate which range of the finger has overlapped on the imaging sensor 107 to generate a fingerprint image for authentication at the time of contact for authentication. As a result, it is possible to narrow down which area of the fingerprint image for authentication corresponds to a fingerprint image for registration in registration database. For example, as illustrated in FIG. 20, in a case where the center of gravity position of the contact of the finger is on the left side of the imaging sensor 107, it can be estimated that the right side of the finger overlaps on the imaging sensor 107 to generate the fingerprint image for authentication. Therefore, it is possible to improve the efficiency of authentication processing by preferentially collating an area on the right side among areas in the registration database.

Moreover, by detecting the center of gravity position of the contact of the finger, a region of the finger whose fingerprint image has been generated and a region of the finger whose fingerprint image has not been generated can also be estimated from the center of gravity position, so that it is also possible to prompt the user to bring the finger into contact with the contact surface T in order to generate a fingerprint image of the region of the finger.

Note that in a case where a strain gauge is used as the pressing sensor 108, the center of gravity position cannot be detected unlike a case where the electrostatic capacitance sensor is used, but if an arrangement is made as illustrated in FIGS. 2 and 15, it is possible to detect that the finger has conic into contact with which of the upper, lower, left, and right of the imaging sensor 107. As a result, it is possible to narrow down which area of the fingerprint image for authentication corresponds to a fingerprint image for registration in a registration database.

Both the strain gauge and the electrostatic capacitance sensor may be used as the pressing sensor 100 by combining the first embodiment and the third embodiment.

The present technology is configured as described above. Although in the fingerprint, the shape of the ridge changes according to the degree of pressing the finger, the accuracy of the fingerprint authentication can be improved by using the degree of pressing as the contact information for the authentication as in the present technology and comparing the fingerprint images of the same degree of pressing between at the time of registration and at the time of authentication. Therefore, even when a contact mode of the finger as the part of the living body is different between at the time of registration and at the time of authentication, highly accurate biometric authentication can be performed.

Furthermore, the authentication processing can be speeded up by performing collation preferentially from the fingerprint image for registration generated at the same degree of pressing as the degree of pressing in contact for authentication. Furthermore, in a case where the degree of pressing the finger is not appropriate at the time of authentication, it is possible to give feedback to the user and guide the user to contact at an appropriate degree of pressing.

Furthermore, by registering the fingerprint image for each of a plurality of degrees of pressing at the time of registration, the feature information can be normalized by associating the degree of pressing with a change in the ridge, and the feature information can be generated without depending on the degree of pressing. Furthermore, by registering the fingerprint image for each of the plurality of degrees of pressing at the time of registration, variations of the degree or pressing assumed at the time of authentication can be secured, so that authentication accuracy can be improved without detecting the degree of pressing at the time of authentication. Furthermore, in a case where a device used for registration and a device used for authentication are different from each other, if the degree of pressing is not detected at the time of authentication, the device to be used for authentication can be simplified, and power consumption can be reduced.

Furthermore, as a size of the device 100 becomes smaller, the imaging sensor 107 for generating a fingerprint image becomes smaller, and a fingerprint image of the entire finger can be efficiently generated even in a case where the fingerprint image of the entire finger cannot be generated by single contact of the finger.

Furthermore, the degree of pressing the finger against the contact surface T itself can also be used as the feature information. Since each person has individuality in a pressing manner, for example, a person tends to press strongly, and a person tends to press weakly, the authentication processing can be performed using the degree of pressing itself as the feature information. Moreover, which part of the contact surface T the finger is to be brought into contact with can also be used as the feature information.

<4. Modifications>

Although the embodiments of the present technology have been specifically described above, the present technology is not limited to the embodiments described above, and various kinds of modifications on based on the technical idea of the present technology are possible.

Authentication based on an image may use a vein pattern in addition to a fingerprint. Furthermore, a part of a living body may be a part of a human body other than a finger (arm, leg, head, torso, iris of the eye, and the like).

In a case where a device 100 that performs registration processing and a device 100 that performs authentication processing are different from each other, the device 100 that performs the authentication processing does not necessarily need to acquire contact information at the time of authentication such as the degree of pressing. Furthermore, even if the device 100 that performs the registration processing and the device 100 that performs the authentication processing are the same, it is not necessary to acquire the contact information at the time of contact for authentication when contact for authentication is performed. In a case where the contact information at the time of authentication is not detected, the authentication processing is performed by comparing a fingerprint image for authentication with a fingerprint image for registration in any level of the degree of pressing in a registration database. Therefore, in the present technology, the authentication processing can be performed with a combination of only the fingerprint images, a combination of the fingerprint images and the degree of pressing as the contact information, a combination of the fingerprint images and center of gravity position information as the contact information, or a combination of the fingerprint images, the degree of pressing as the contact information, and the center of gravity position information.

In a case where the softness of the finger can be estimated from a change in the shape of a ridge due to a difference in the degree of pressing, the softness can also be used as feature information. The use of the softness of the finger as information for authentication makes it possible to distinguish between a false finger including plastic, rubber, or the like and a real finger and to improve the accuracy of authentication.

Furthermore, in a case where a blood flow volume can be detected from the fingerprint image, the blood flow volume can also be used as the feature information. For example, the use of the blood flow volume as the feature information makes it possible to distinguish between a false finger including plastic, rubber, or the like and a real finger and improve the accuracy of authentication.

Furthermore, since a heart rate of a user can be acquired on the basis of the blood flow volume, a state of the user can be estimated from the heart rate. For example, the state of the user is estimated in a procedure that requires the authentication processing (transfer procedure or the like), it is possible to determine whether to execute the procedure although the authentication has been successful. In a case where since the heart rate is a predetermined value or more, the user is not in a normal state (being irritated, being deceived, and the like), for example, the procedure is not performed even if the authentication is successful. This can be used for measures against bank transfer fraud and the like.

Furthermore, in a case where the color of the finger can be detected from the fingerprint image, the color or the finger can also be used as the feature information. Since the color of a human finger changes by being pressed, the use of the change as the feature information makes it possible to distinguish between a false finger including plastic, rubber or the like whose color does not change and a real finger and to improve the accuracy of authentication.

Moreover, the accuracy of authentication may be improved by using in parallel biometric information that can be detected by a body temperature sensor, a heart rate sensor, or the like included in a wristwatch-type wearable device.

The present technology can also have the following configurations.

(1)

An information processing apparatus including: a processing unit that performs living body-related processing on the basis of an image of a part of a living body of a user, the image being generated in a state in which the part of the living body is in contact with a contact surface, and contact information acquired in the state in which the part of the living body is in contact with the contact surface.

(2)

The information processing apparatus according to (1), in which the living body-related processing is registration processing of a plurality of images for registration generated by a plurality of times of contact for registration with the contact surface.

(3)

The information processing apparatus according to (2), in which each of the plurality of images for registration is an image of a different region of the part of the living body.

(4)

The information processing apparatus according to (2) or (4), in which the plurality of images for registration is registered while corresponding to a plurality of pieces of contact information at the time of registration for each of the plurality of times of contact for registration.

(5)

The information processing apparatus according to any of (2) to (4), in which the living body-related processing is biometric authentication processing using the image for registration and an image for authentication generated by contact for authentication with the contact surface after the registration processing is completed.

(6)

The information processing apparatus according to (5), in which the biometric authentication processing is performed by comparing the image for registration registered while corresponding to the contact information at the time of registration that is the same as contact information at the time of authentication in the contact for authentication with the image for authentication.

(7)

The information processing apparatus according to (5) or (6), in which the biometric authentication processing is performed by comparing feature information acquired from the image for registration registered in the registration processing with feature information acquired from the image for authentication.

(8)

The information processing apparatus according to any of (1) to (7), in which the contact information is pressing force by the part of the living body against the contact surface.

(9)

The information processing apparatus according to any of (1) to (8), in which the contact information is an area of contact of the part of the living body with respect to the contact surface.

(10)

The information processing apparatus according to any of (1) to (9), in which the contact information is a center of gravity position of contact with the contact surface by the part of the living body.

(11)

The information processing apparatus according to any of (1) to (10), in which the contact information is shear force by the part of the living body against the contact surface.

(12)

The information processing apparatus according to any of (1) to (11), further including: a UI processing unit that provides information to the user so that the user brings the part of the living body into contact with the contact surface a plurality of times in a different mode.

(13)

The information processing apparatus according to (12), in which the information processing apparatus provides the information to the user so that the user generates the images of a plurality of regions in the part of the living body.

(14)

The information processing apparatus according to (12) or (13), in which the information is provided to the user so that the user brings the part of the living body into contact with the contact surface at a different degree of pressing.

(15)

The information processing apparatus according to any of (1) to (14), in which a center of gravity position of contact of the part of the living body with the contact surface is detected, and the image for registration to be compared with the image for authentication in the biometric authentication processing is determined.

(16)

The information processing apparatus according to any of (1) to (15), in which the part of the living body is a finger, and the image is a fingerprint image.

(17)

An information processing method of performing living body-related processing on the basis of an image of a part of a living body of a user, the image being generated in a state in which the part of the living body is in contact with a contact surface, and the contact information acquired in a state in which the part of the living body is in contact with the contact surface.

(18)

An information processing program causing a computer to execute an information processing method of performing living body-related processing on the basis of an image of a part of a living body of a user, the image being generated in a state in which the part of the living body is in contact with a contact surface, and the contact information acquired in a state in which the part of the living body is in contact with the contact surface.

### REFERENCE SIGNS LIST

100 Device
200 Information processing apparatus
203 Registration processing unit
204 Authentication processing unit
205 UI processing unit
T Contact surface

1. An information processing apparatus comprising:
a processing unit that performs living body-related processing on a basis of an image of a part of a living body of a user, the image being generated in a state in which the part of the living body is in contact with a contact surface, and contact information acquired in the state in which the part of the living body is in contact with the contact surface.

2. The information processing apparatus according to claim 1, wherein
the living body-related processing is registration processing of a plurality of images for registration generated by a plurality of times of contact for registration with the contact surface.

3. The information processing apparatus according to claim 2, wherein
each of the plurality of images for registration is an image of a different region of the part of the living body.

4. The information processing apparatus according to claim 2, wherein
the plurality of images for registration is registered while corresponding to a plurality of pieces of contact information at the time of registration for each of the plurality of times of contact for registration.

5. The information processing apparatus according to claim 2, wherein
the living body-related processing is biometric authentication processing using the image for registration and an image for authentication generated by contact for authentication with the contact surface after the registration processing is completed.

6. The information processing apparatus according to claim 5, wherein
the biometric authentication processing is performed by comparing the image for registration registered while corresponding to the contact information at the time of registration that is the same as contact information at the time of authentication in the contact for authentication with the image for authentication.

7. The information processing apparatus according to claim 5, wherein
the biometric authentication processing is performed by comparing feature information acquired from the image for registration registered in the registration processing with feature information acquired from the image for authentication.

8. The information processing apparatus according to claim 1, wherein
the contact information is pressing force by the part of the living body against the contact surface.

9. The information processing apparatus according to claim 1, wherein
the contact information is an area of contact of the part of the living body with respect to the contact surface.

10. The information processing apparatus according to claim 1, wherein
the contact information is a center of gravity position of contact with the contact surface by the part of the living body.

11. The information processing apparatus according to claim 1, wherein
the contact information is shear force by the part of the living body against the contact surface.

12. The information processing apparatus according to claim 1, further comprising:
a UI processing unit that provides information to the user so that the user brings the part of the living body into contact with the contact surface a plurality of times in a different mode.

13. The information processing apparatus according to claim 12, wherein
the information processing apparatus provides the information to the user so that the user generates the images of a plurality of regions in the part of the living body.

14. The information processing apparatus according to claim 12, wherein
the information is provided to the user so that the user brings the part of the living body into contact with the contact surface at a different degree of pressing.

15. The information processing apparatus according to claim 1, wherein
a center of gravity position of contact of the part of the living body with the contact surface is detected, and the image for registration to be compared with the image for authentication in the biometric authentication processing is determined.

16. The information processing apparatus according to claim 1, wherein
the part of the living body is a finger, and the image is a fingerprint image.

17. An information processing method of performing
living body-related processing on a basis of an image of a part of a living body of a user, the image being generated in a state in which the part of the living body is in contact with a contact surface, and the contact information acquired in a state in which the part of the living body is in contact with the contact surface.

18. An information processing program causing a computer to execute an information processing method of performing
living body-related processing on a basis of an image of a part of a living body of a user, the image being generated in a state in which the part of the living body is in contact with a contact surface, and the contact information acquired is a state in which the part of the living body is in contact with the contact surface.
